(19) United States
(12) Patent Application Publication
(10) Pub. No.: US 2023/0051939 A1
(43) Pub. Date: Feb. 16, 2023

(54) PHYSIOLOGICAL MONITORING APPARATUS AND PHYSIOLOGICAL MONITORING METHOD

(71) Applicant: MEDIATEK INC., Hsinchu City (TW)

(72) Inventors: Hung-Chih CHIU, Hsinchu City (TW); Shu-Yu HSU, Hsinchu City (TW); Wei-Han LIU, Hsinchu City (TW)

(21) Appl. No.: 17/399,201

(22) Filed: Aug. 11, 2021

(51) Int. Cl. A61B 5/00; A61B 5/024; A61B 5/1455; A61B 5/08; A61B 5/0205; A61B 5/113; A61B 5/318

(52) U.S. Cl. CPC A61B 5/4818; A61B 5/02433; A61B 5/14551; A61B 5/0816; A61B 5/0205; A61B 5/113; A61B 5/318; A61B 5/7203

(57) ABSTRACT

A physiological monitoring device is provided and includes a physiological sensing device, a first PPG sensor, a vital signs detector, and a PPG controller. The physiological sensing device senses at least one physiological feature of a subject to generate at least one sensing signal. The first PPG sensor senses pulses of a blood vessel of the subject to generate a first PPG signal when the first PPG sensor is activated. The vital signs detector obtains vital signs data according to the at least one sensing signal. The PPG controller detects whether a specific event is happening to the subject according to the vital signs data. In response to detecting that the specific event is happening to the subject, the PPG controller activates the first PPG sensor. The physiological monitoring apparatus obtains a blood oxygen level of the subject according to the first PPG signal.

### BACKGROUND OF THE INVENTION

### Field of the Invention

The invention relates to a physiological monitoring device, and more particularly to a physiological monitoring device which can measure the blood oxygen level of a user while consuming less power during the monitoring period.

### Description of the Related Art

Wearable devices are trending these days. Some wearable devices are capable of monitoring and tracking medical and health information for users, such as blood oxygen level, electrocardiography (ECG), photoplethysmogram (PPG), heart rate, and blood pressure. These wearable devices may enable continuous healthcare monitoring, even when the users are feeling well or are in normal health. This constant monitoring, however, increases power consumption. In particularly, the light sources used in the monitoring of blood oxygen level use a lot of power. In order to reduce power consumption, some healthcare monitoring functions, such as blood oxygen level monitoring, are deactivated by default unless the users activate these functions by themselves. In this case, when the users suddenly become uncomfortable or the users' bodily conditions suddenly appear abnormal, the wearable devices cannot record the vital-sign signals or values in time, which limits the capability of these wearable devices.

### BRIEF SUMMARY OF THE INVENTION

An exemplary embodiment of a physiological monitoring device. The physiological monitoring device comprises a physiological sensing device, a first photoplethysmogram (PPG) sensor, a vital signs detector, and a PPG controller. The physiological sensing device is configured to sense at least one physiological feature of a subject to generate at least one sensing signal. The first PPG sensor is configured to sense pulses of a blood vessel of the subject to generate a first PPG signal when the first PPG sensor is activated. The vital signs detector is configured to receive the at least one sensing signal and obtain vital signs data according to the at least one sensing signal. The PPG controller is configured to detect whether a specific event is happening to the subject according to the vital signs data. In response to detecting that the specific event is happening to the subject, the PPG controller activates the first PPG sensor. The physiological monitoring apparatus obtains a blood oxygen level of the subject according to the first PPG signal.

An exemplary embodiment of a physiological monitoring method is provided. The physiological monitoring method comprises the steps of sensing at least one physiological feature of a subject to generate at least one sensing signal; obtaining vital signs data according to the at least one sensing signal; detecting whether a specific event is happening to the subject according to the vital signs data; in response to detecting that the specific event is happening to the subject, activating a PPG sensor to sense pulses of a blood vessel of the subject and generate a first PPG signal according to the sensed pulses; and obtains a blood oxygen level of the subject according to the first PPG signal.

A detailed description is given in the following embodiments with reference to the accompanying drawings.

### BRIEF DESCRIPTION OF THE DRAWINGS

The invention can be more fully understood by reading the subsequent detailed description and examples with references made to the accompanying drawings, wherein:

FIG. 1 shows one exemplary embodiment of a physiological monitoring apparatus;

FIG. 2 shows another exemplary embodiment of a physiological monitoring apparatus;

FIG. 3 shows variations in SpO2 percentage in cases where the user experiences no apnea;

FIGS. 4A-4B show variations in SpO2 percentage in cases where another user (such as another male) experiences severe sleep apnea;

FIG. 5 is a schematic diagram showing an activated/deactivated state of a PPG sensor based on controlling of a PPG controller according to an exemplary embodiment;

FIG. 6 is a schematic diagram showing an X-axis component, a Y-axis component, and a Z-axis component of a sensing signal generated by a motion sensor according to an exemplary embodiment;

FIGS. 7A-7C are schematic diagrams showing a processed sensing signal and a respiratory signal according to an exemplary embodiment;

FIG. 8 shows another exemplary embodiment of a physiological monitoring apparatus; and

FIG. 9 shows an exemplary embodiment of a physiological monitoring apparatus.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

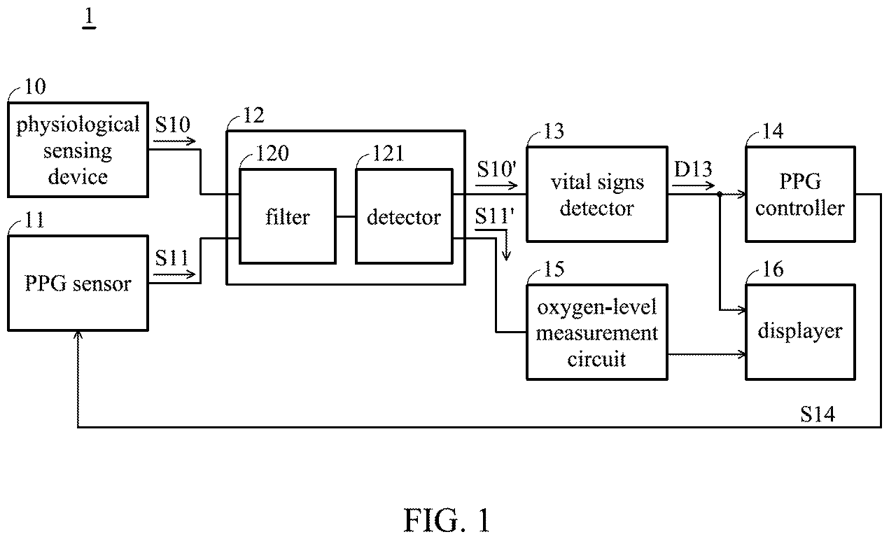

FIG. 1 shows one exemplary embodiment of a physiological monitoring apparatus. As shown in FIG. 1, a physiological monitoring apparatus 1 comprises a physiological sensing device 10, a photoplethysmogram (PPG) sensor 11, a pre-processor 12, a vital signs detector 13, a PPG controller 14, an oxygen-level measurement circuit 15, and a displayer 16. The physiological monitoring apparatus 1 can be a wearable device or a physiological monitor with a blood oxygen monitoring function and another or other physiological monitoring functions for monitoring at least one vital sign of a subject (such as a user) who wears, holds, or contacts the physiological sensing device 10 and the PPG sensor 11. The at least one vital sign of the user comprises, for example, one or more of the following: heart rate, respiration rate, breathing activity, state of motion, and apnea event of the user. The physiological sensing device 10 may comprise at least one physiological-feature detector which operates to sense at least one physiological feature of the user, such as electrocardiography (ECG), photoplethysmogram (PPG), and/or motions of the user which are related to the user's heart rate, respiration rate, state of motion, and/or apnea event. The physiological sensing device 10 generates at least one sensing signal S10 based on the sensed results.

The pre-processor 12 receives the sensing signal S10. The pre-processor 12 then processes the sensing signal S10 by performing a filter operation and a motion-artifact removal operation on the sensing signal S10 to generate a processed sensing signal S10. In an embodiment, the pre-processor 12 comprises a filter 120 which performs the filter operation to filter a direct-current component and the high-frequency noise from the sensing signal S10. Moreover, the pre-processor 12 further comprises a detector 121 which detects a motion artifact and performs the motion-artifact removal operation to remove at least one signal section, which corresponds to the motion artifact of the user, from the sensing signal S10.

The processed sensing signal S10′ is provided to the vital signs detector 13. The vital signs detector 13 receives the processed sensing signal S10′ and obtains vital signs data D13 according to the processed sensing signal S10′. The vital signs data comprises information related to the heart rate, the respiration rate, the breathing activity, and/or the state of motion of the user. The vital signs detector 13 provides the vital signs data D13 to the PPG controller 14.

When receiving the vital signs data D13, the PPG controller 14 detects whether a specific event is happening to the user according to the vital signs data D13. The PPG controller 14 generates a control signal S14 according to the detection result. When the PPG controller 14 detects that the specific event is happening to the user according to the vital signs data D13, the PPG controller 14 activates the PPG sensor 11 through the control signal S14. In the embodiments, the PPG controller 14 detects that the specific event is happening to the user when the user experiences an apnea event, the heart rate of the user is not in a normal range, or the blood pressure of the user is not in a normal range.

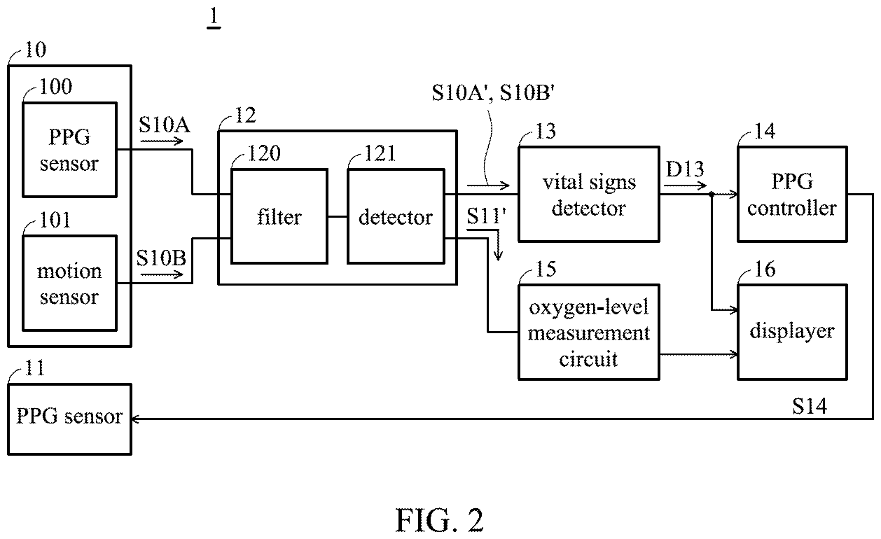

The PPG sensor 11 comprises a red (R) light source and an infrared (IR) light source. When the PPG sensor 11 is activated through the control signal S14, the PPG sensor 11 illuminates the skin of user by red light beams and infrared light beams which are emitted from the red (R) light source and the infrared (IR) light source respectively. The light beams, which are emitted from the red (R) light source and the infrared (IR) light source, travel through the tissue and blood under the skin and then collected in the PPG sensor 11. The PPG sensor 11 detects the changes in light absorption of the blood under the skin according to the collected light means for sensing pulses of the blood vessel of the user. The PPG sensor 11 generates a PPG signal S11 according to the amount of received red (R) light beams and the infrared (IR) light beams. Because the deoxyhemoglobin (Hb) and the oxyhemoglobin (HbO2) in the blood have different capacities for the red (R) light and the infrared (IR) light having different wavelengths, the PPG signal S11 is related to the amount of the deoxyhemoglobin (Hb) and the amount of the oxyhemoglobin (HbO2) in the blood. The PPG signal S11 is provided to the pre-processor 12. The pre-processor 12 processes the PPG signal S11 by performing a filter operation and a motion-artifact removal operation on the PPG signal S11. In an embodiment, the motion artifact can be detected by a motion sensor, such as a motion sensor 101 shown in FIG. 2. The processed PPG signal S11′ is provided to the oxygen-level measurement circuit 15. The oxygen-level measurement circuit 15 obtains the blood oxygen level of the user according to the PPG signal S11. In the embodiment, the blood oxygen level is represented by an oxygen saturation (SpO2) percentage. The oxygen-level measurement circuit 15 provides the oxygen saturation percentage to the displayer 16, and the displayer 16 shows the oxygen saturation percentage on the screen.

In an embodiment, when the PPG sensor 11 is activated for a predetermined period of time, the PPG sensor 11 is deactivated. For example, the predetermined period of time is in a range of 10-60 sec.

According to the embodiment, the PPG sensor 11 for the blood oxygen monitoring function is not always activated. The PPG sensor 11 is deactivated initially and activated automatically when a specific event is happening to the user. Accordingly, when the user suddenly becomes uncomfortable or the user's bodily conditions suddenly appear abnormal, the physiological monitoring apparatus can monitor the blood oxygen level in real time.

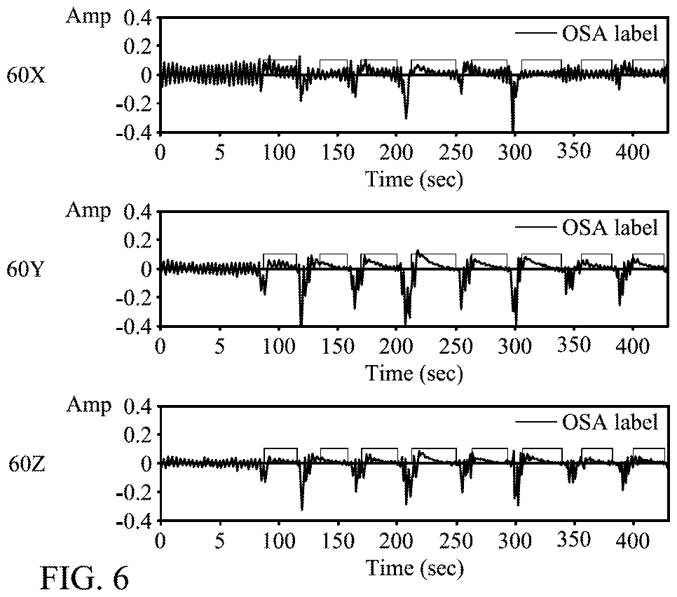

In an embodiment, as shown in FIG. 2, the physiological sensing device 10 comprises a PPG sensor 100 and a motion sensor 101. The PPG sensor 100 comprises a green (G) light source, a red (R) light source, or an infrared (IR) light source. The PPG sensor 100 illuminates the skin of user (for example, the skin of the right wrist) by the red (R) light source, the green (G) light source, or the infrared (IR) light source and then collected in the PPG sensor 100. The PPG sensor 100 detects the changes in light absorption of the blood under the skin for sensing the pulses of the blood vessel of the user. The PPG sensor 100 generates a sensing signal (also referred to as a PPG signal) S10A based on the measured changes. The motion sensor 101 is disposed on a specific portion of the body of the user, such as one arm, one wrist, or one leg of the user, to sense the motion or activity of the user and generate a sensing signal (also referred to as a motion signal) S10B. In an embodiment, the motion sensor 101 comprises an accelerometer, and the sensing signal S10B comprises an X-axis component, a Y-axis component, and a Z-axis component (shown in FIG. 6). The pre-processor 12 receives the sensing signals S10A and S10B. The pre-processor 12 processes the sensing signals S10A and S10B by performing a filter operation and a motion-artifact removal operation on the sensing signals S10A and 10B. The processed sensing signal (also referred to as a processed PPG signal) S10A′ and the processed sensing signal (also referred to as a processed motion signal) S10B′ are provided to the vital signs detector 13.

In an embodiment where the PPG sensor 100 emits red light beams or infrared light beams, the PPG sensor 100 and the PPG sensor 11 may share the same red light source and the infrared light source.

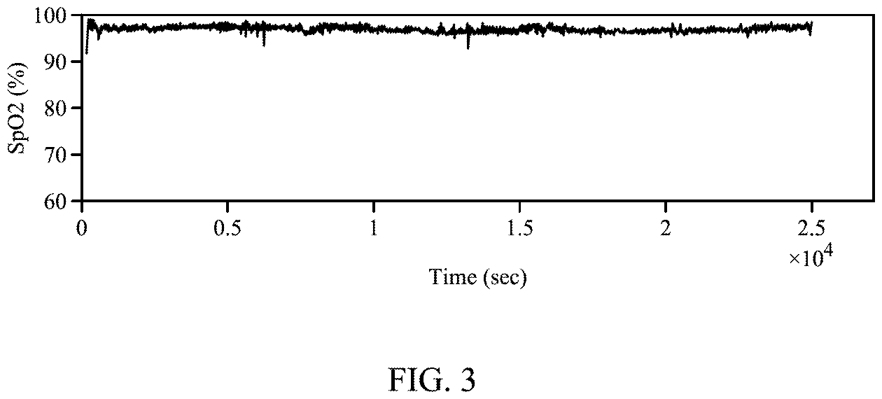

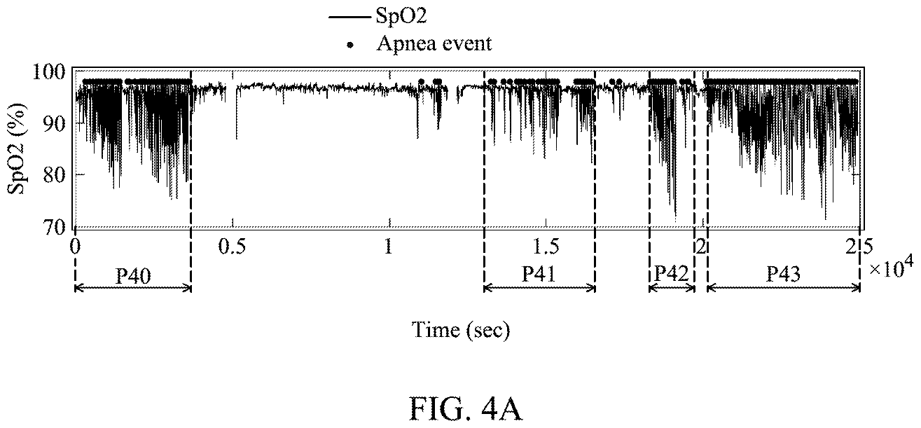

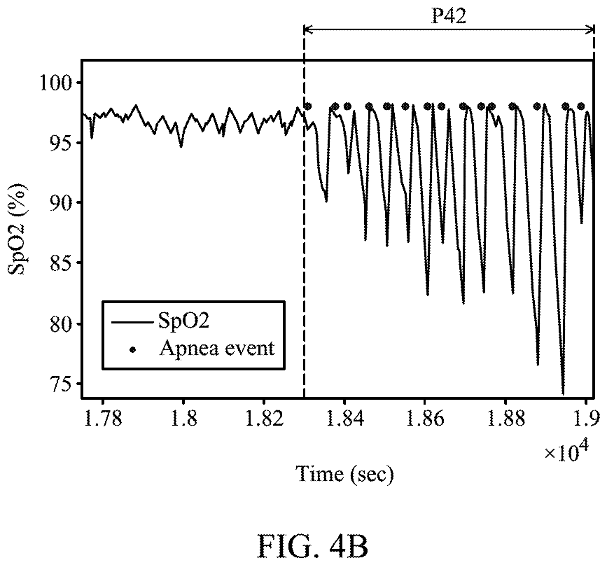

In the following paragraphs, it is assumed that the specific event is an apnea event (that is, the specific event happens when the user experiences an apnea event) for explaining the invention of the present application. FIG. 3 shows variations in SpO2 percentage in cases where a user (such as a male) experiences no sleep apnea, while FIG. 4A shows variations in SpO2 percentage in cases where another user (such as another male) experiences severe sleep apnea. In FIGS. 3 and 4A, the variations in SpO2 percentages are represented by respective waveforms. In general, the normal oxygen saturation (SpO2) percentage is 94%˜100%. Referring to FIG. 3, the SpO2 percentage does not change much and is 94%-100% most of the time during sleep. However, referring to FIG. 4A, during some periods of time, such as P40-P43, the SpO2 percentage changes greatly and is lower than the lower threshold (94%) of the normal range. The user shown in FIG. 4A is also monitored by a sleep apnea monitoring device, such as a polysomnography (PSG) device. Each sign “ ” represents an apnea event. FIG. 4B shows an enlarged view of the waveform of the variations in SpO2 percentage and the apnea-event signs during a period P42. As shown in FIGS. 4A-4B, the occurrence of apnea events causes a decrease in the oxygen saturation which is a warning sign of physical problems. Thus, an apnea event can serve as an important factor for activating the blood oxygen monitoring function (that is, activating the PPG sensor 11).

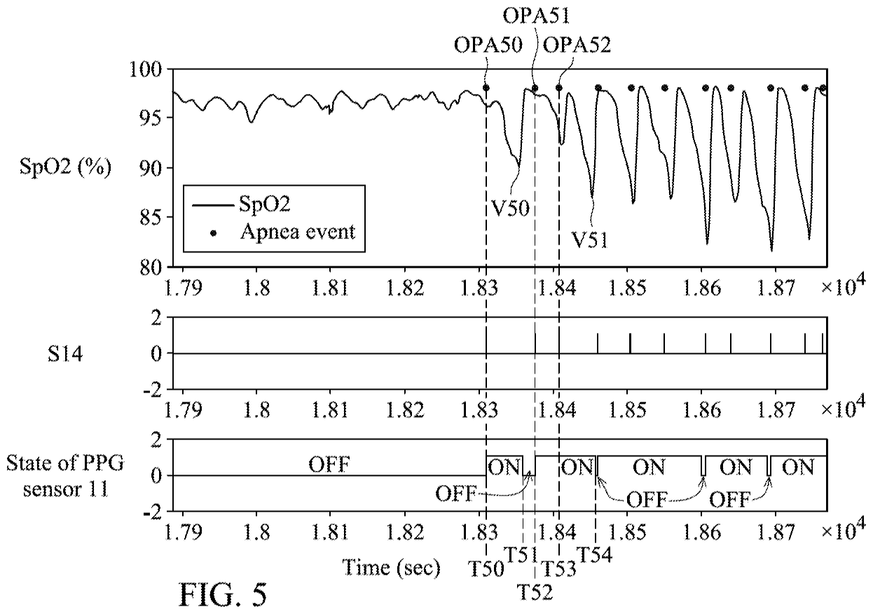

FIG. 5 is a schematic diagram showing the activated/deactivated state of the PPG sensor 11 based on the controlling of the PPG controller 14. The waveform of the SpO2 percentage shown in FIG. 5 is a portion of the waveform shown in FIG. 4B. In FIG. 5, the activated state of the PPG sensor 11 is represented by “ON”, while the deactivated state thereof is represented by “OFF”. Referring to FIG. 5, the PPG controller 14 detects that an apnea event OPA50 is happening to the user according to the vital signs data D13 at the time point T50, the PPG controller 14 generates the control signal S14 with a pulse to activate the PPG sensor 11. When the PPG sensor 11 is activated for a period of time and no apnea event is further detected during the period of time, the PPG sensor 11 is deactivated at the time point T51. In an embodiment, the period of time between the time points T50 and T51 is predetermined, for example, the predetermined period of time is in a range of 10-60 sec. In another embodiment, the time point T51 is present near the minimum value of the SpO2 percentage which is present after the time point T50. As shown in FIG. 5, in detail, the minimum value of the SpO2 percentage which is present after the time point T50 is the value at the valley V50 of the waveform of the SpO2 percentage. In a case where an apnea event is detected during the activated state of the PPG sensor 11, the PPG sensor 11 is activated continuously. For example, during the period when the PPG sensor 11 is activated at the time point T52 in response to the apnea event OPA51, another apnea event OPA52 is further detected at the time T53. In this case, the PPG sensor 11 is activated continuously until the time point T54. Similarly, the period of time between the time points T53 and T54 is the predetermined period, or the time point T54 is present near the valley of the waveform of the SpO2 percentage which is present after the time point T53.

According to the above embodiment, the PPG sensor 11 is activated in response to apnea events instead of be always activated. Thus, compared to the case in which a PPG sensor is always activated during sleep, the power consumption of the physiological monitoring apparatus 1 can be saved due to the controllable activated state of the PPG sensor 11.

In the following paragraphs, how to detect whether an apnea event occurs during sleep will be described.

According to an embodiment, an apnea event can be detected according to the breathing activity and the state of motion of the user. FIG. 6 is a schematic diagram showing an X-axis component, a Y-axis component, and a Z-axis component of the processed sensing signal (the processed motion signal) S10B′ according to an exemplary embodiment. The diagram 60X shows the X-axis component of the processed sensing signal S10B′, the diagram 60Y shows the Y-axis component of the processed sensing signal S10B′, and the diagram 60Z shows the Z-axis component of the processed sensing signal S10B′. The amplitudes of the X-axis, Y-axis, and Z-axis components of the processed sensing signal S10B′ indicate the state of motion of the user. In order to demonstrate the operation of the physiological monitoring apparatus 1, while the user is monitored by the physiological monitoring apparatus 1 during sleeping, the user is also monitored by a sleep apnea monitoring device which generates event labels OSA indicating apnea events. Referring to FIG. 6, the above event labels OSA are shown on the time axes of the diagrams 60X, 60Y, and 60Z, and each event label spans a period of time. Referring to FIG. 6, when an apnea event occurs, the amplitudes of the X-axis, Y-axis, and Z-axis components of the processed sensing signal S10B′ are decreased. Thus, whether an apnea event occurs can be determined based on the amplitudes of the X-axis, Y-axis, and Z-axis components of the processed sensing signal S10B′.

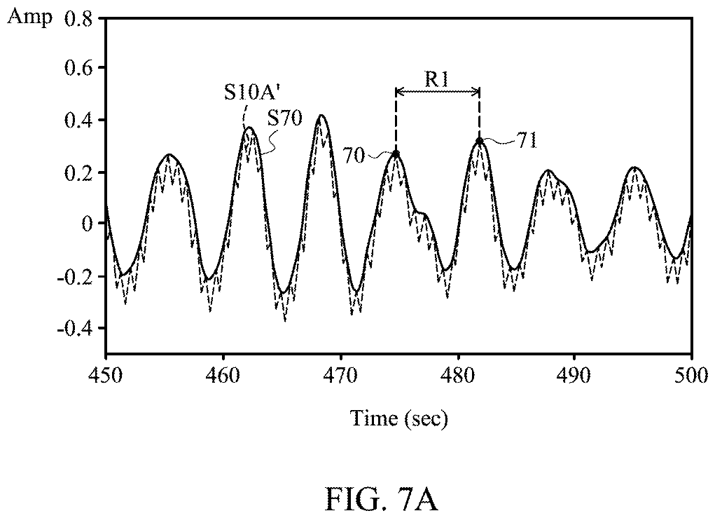

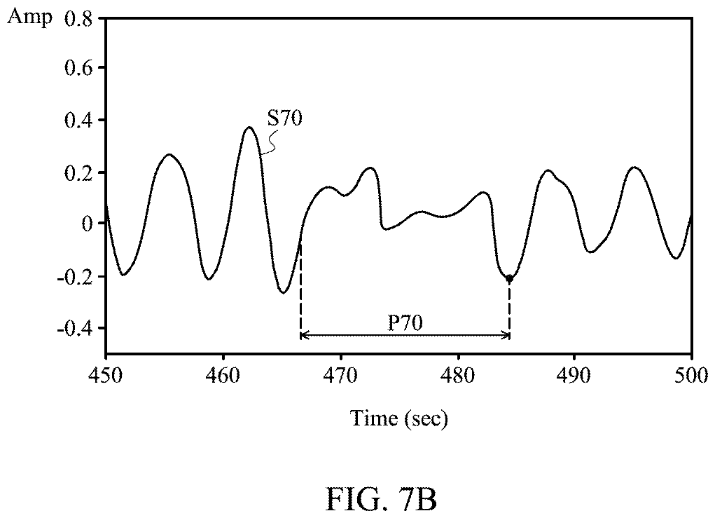

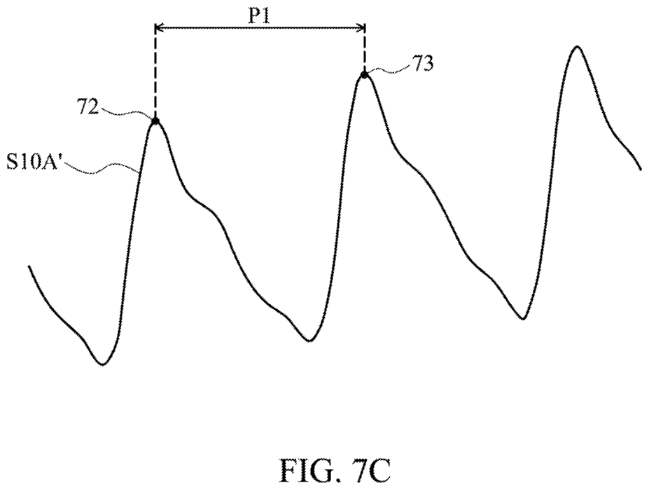

FIG. 7A is a schematic diagram showing the processed sensing signal (the processed PPG signal) S10A′ obtained from the PPG sensor 100. Since the venous blood flow is aided by breathing activity, a PPG signal comprises a component related to a respiratory signal. As shown in FIG. 7A, the vital signs detector 13 takes the envelope of the processed sensing signal S10A′ as a respiratory signal S70 and further estimates the amplitude of the respiratory signal S70. The estimated amplitude of the respiratory signal S70 represents the breathing activity. In general, an apnea event occurs when the breathing activity is stopped. Referring to FIG. 7B, for example, during the period P70, the estimated amplitude of the respiratory signal S70 is decreased while an apnea event occurs. Thus, whether an apnea event occurs can be determined based on the respiratory signal S70 derived from the processed sensing signal S10A′.

According to the above embodiment, the vital signs detector 13 receives the processed sensing signal S10A′ and obtains the respiratory signal S70 according to the processed sensing signal S10′ A. The vital signs detector 13 estimates the amplitude of the respiratory signal S70. The vital signs detector 13 further receives the processed sensing signal S10B′ and estimates the amplitudes of the X-axis, Y-axis, and Z-axis components of the processed sensing signal S10B′. The vital signs detector 13 obtains the vital signs data D13 according to the estimated amplitude of the respiratory signal S70 and the estimated amplitudes of the X-axis, Y-axis, and Z-axis components of the processed sensing signal S10B′.

The PPG controller 14 receives the vital signs data D13 to retrieve the estimated amplitude of the respiratory signal S70 and the estimated amplitudes of the X-axis, Y-axis, and Z-axis components of the processed sensing signal S10B′. The PPG controller 14 determines whether the estimated amplitude of the respiratory signal S70 is less than a predetermined threshold to generate a determination result and further determines whether the estimated amplitudes of the X-axis, Y-axis, and Z-axis components of the processed sensing signal S10B′ are less than another predetermined threshold to generate another determination result. The PPG controller 14 detects whether an apnea event occurs according to the determination results. For example, when the estimated amplitude of the respiratory signal S70 is less than the corresponding predetermined threshold and/or the estimated amplitudes of the -axis, Y-axis, and Z-axis components of the processed sensing signal S10B′ are less than the corresponding predetermined threshold, the PPG controller 14 detects that an apnea event is happening to the user and generates the control signal S14 with a pulse to activate the PPG sensor 11. In an embodiment, the PPG sensor 100 is deactivated while the PPG sensor 11 is activated.

According to another embodiment, a specific event may be detected according to the heart rate of the user. FIG. 7C shows a partial enlarged view of the processed sensing signal (the processed PPG signal) S10A′. Referring to FIG. 7C, there are several peaks on the processed sensing signal S10A′. The time interval between two adjacent peaks of the processed sensing signal S10A′ can be taken for estimation of the heart rate of the user. In the embodiment, when the vital signs detector 13 receives the processed sensing signal S10A′, the vital signs detector 13 detects the peaks of the processed sensing signal S10′ and calculates a time interval between two adjacent peaks, for example the time interval P1 in seconds between two adjacent peaks 72 and 73 shown in FIG. 7C. The vital signs detector 13 then estimates the heart rate of the user according to the calculated time interval and obtains the vital signs data D13 according to the estimated heart rate. For example, the vital signs detector 13 estimates the heart rate (bpm) by dividing 60 sec by the calculated time interval (in seconds). In the embodiment, the PPG controller 14 receives the vital signs data D13 to retrieve the estimated heart rate and determines whether the estimated heart rate is within a normal range, such as a rage of 60-100 bpm. When the estimated heart rate is not within the normal range, the PPG controller 14 detects that a specific event is happening to the user and generates the control signal S14 with a pulse to activate the PPG sensor 11. In an embodiment, the vital signs detector 13 further provides the vital signs data D13 to the displayer 16, and the displayer 16 shows the heart rate on the screen.

According to another embodiment, a specific event may be detected according to the respiration rate of the user. Referring to FIG. 7A, there are several peaks on the respiratory signal S70. The time interval between two adjacent peaks of the respiratory signal S70 can be taken for estimation of the respiration rate of the user. In the embodiment, when the vital signs detector 13 obtains the respiratory signal S70 according to the processed sensing signal S10A′, the vital signs detector 13 detects the peaks of the respiratory signal S70 and calculates a time interval between two adjacent peaks, for example the time interval R1 in minutes between two adjacent peaks 70 and 71 shown in FIG. 7A. The vital signs detector 13 then estimates the respiration rate of the user according to the calculated time interval and obtains the vital signs data D13 according to the estimated respiration rate. For example, the vital signs detector 13 estimates the respiration rate (in breaths per minute). In the embodiment, the PPG controller 14 receives the vital signs data D13 to retrieve the estimated respiration rate and determines whether the estimated respiration rate is within a normal range, such as a rage of 12-20 breaths per minute. When the estimated respiration rate is not within the normal range, the PPG controller 14 detects that a specific event is happening to the user and generates the control signal S14 with a pulse to activate the PPG sensor 11. In an embodiment, the vital signs detector 13 further provides the vital signs data D13 to the displayer 16, and the displayer 16 shows the respiration rate on the screen.

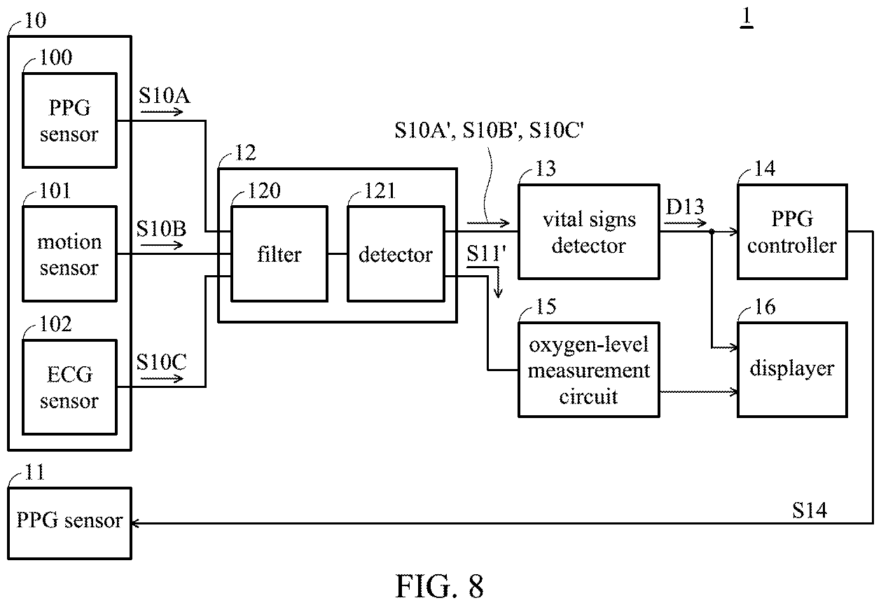

FIG. 8 shows another exemplary embodiment of a physiological monitoring apparatus. Referring to FIG. 8, the physiological sensing device 10 further comprises an electrocardiography (ECG) sensor 102. When the ECG sensor 102 is activated to sense the electrical activity of the heart of the user through electrodes contacting the skin of the user, the ECG sensor 102 generates a sensing signal (also referred to as an ECG signal) S10C. The pre-processor 12 receives the sensing signal S10C. The pre-processor 12 then processes the sensing signal S10C by performing a filter operation and a motion-artifact removal operation on the sensing signal S10C to generate a processed sensing signal (also referred to as a processed ECG signal) S10C′. The processed sensing signal S10C′ is provided to the vital signs detector 13. The vital signs 13 detector estimates the heart rate of the user according to the processed sensing signal S10C′ and obtains the vital signs data D13 according to the estimated heart rate. The vital signs data D13 is provided to the PPG controller 14. The PPG controller 14 then retrieves the estimated heart rate from the vital signs data D13 and determines whether the estimated heart rate is within a normal range, such as a rage of 60-100 bpm. When the estimated heart rate is not within the normal range, the PPG controller 14 detects that a specific event is happening to the user and generates the control signal S14 with a pulse to activate the PPG sensor 11.

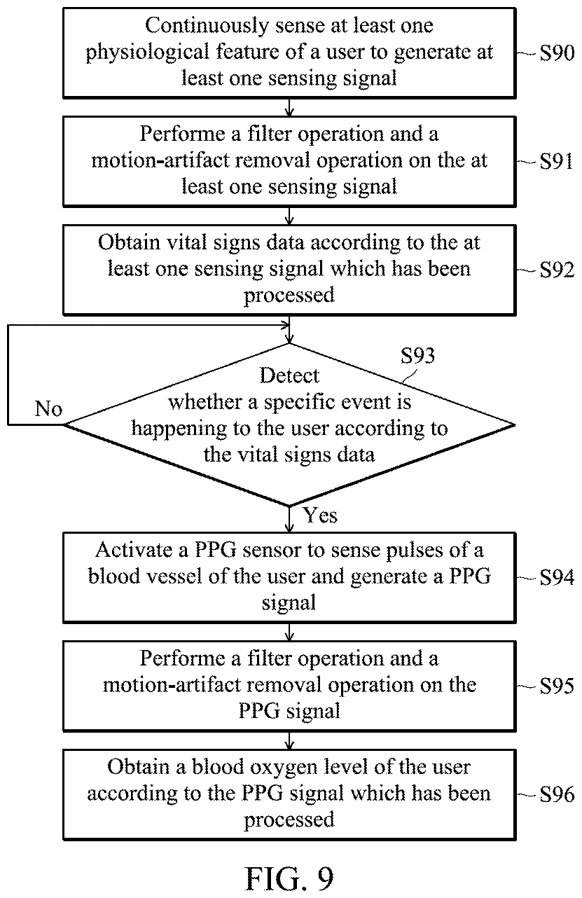

FIG. 9 shows an exemplary embodiment of a physiological monitoring method. The physiological monitoring method will be described by referring to FIGS. 1 and 10. When the physiological monitoring apparatus 1 operates, the physiological sensing device 10 continuously senses at least one physiological feature of a user to generate at least one sensing signal S10 (Step S90). In an embodiment, the least one sensing signal S10 may comprise a PPG signal and a motion. Then, the pre-processor 12 processes the at least one sensing signal by performing a filter operation and a motion-artifact removal operation on the at least one sensing signal S10 (Step S91). The vital signs detector 13 receives the at least one sensing signal S10′ which has been processed by the pre-processor 12 and obtains vital signs data D13 according to the at least one sensing signal S10′ which has been processed (Step S92). The PPG controller 14 receives the vital signs data D13 and detects whether a specific event is happening to the user according to the vital signs data D13 (Step S93). When the specific event is happening to the user (Step S93-Yes), the PPG controller 14 activates the PPG sensor 11 to sense pulses of a blood vessel of the user and generate a PPG signal S11 according to the sensed pulses (Step S94). The pre-processor 12 receives the PPG signal S11 and also processes the PPG signal S11 by performing a filter operation and a motion-artifact removal operation on the PPG signal S11 (Step S95). The oxygen-level measurement circuit 15 receives the PPG signal S11′ which has been processed by the pre-processor 12 and obtains the blood oxygen level of the user according to the PPG signal S11′ which has been processed (Step S96). When the specific event is not happening to the user (Step S93-No), the method continuously performs Step 92 for detecting whether a specific event is happening to the user according in real time.

While the invention has been described by way of example and in terms of the preferred embodiments, it should be understood that the invention is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A physiological monitoring apparatus comprising:
a physiological sensing device configured to sense at least one physiological feature of a subject to generate at least one sensing signal;
a first photoplethysmogram (PPG) sensor configured to sense pulses of a blood vessel of the subject to generate a first PPG signal when the first PPG sensor is activated;
a vital signs detector configured to receive the at least one sensing signal and obtain vital signs data according to the at least one sensing signal; and
a PPG controller configured to detect whether a specific event is happening to the subject according to the vital signs data,
wherein in response to detecting that the specific event is happening to the subject, the PPG controller activates the first PPG sensor, and
wherein the physiological monitoring apparatus obtains a blood oxygen level of the subject according to the first PPG signal.

2. The physiological monitoring apparatus as claimed in claim 1, wherein the first physiological sensing device comprises:
a second PPG sensor configured to sense pulses of a blood vessel of the subject to generate a second PPG signal,
wherein the second PPG signal serves as one of the at least one sensing signal, and
wherein the vital signs data comprises information related to at least one of a heart rate of the user, a respiration rate, and breathing activity of the subject.

3. The physiological monitoring apparatus as claimed in claim 2, wherein the first physiological sensing device further comprises:
an electrocardiography (ECG) sensor configured to sense electrical activity of the heart of the subject and generate an ECG signal,
wherein the ECG signal serves as one of the at least one sensing signal, and
wherein the vital signs data comprises information related to a heart rate of the subject.

4. The physiological monitoring apparatus as claimed in claim 2, wherein the first physiological sensing device further comprises:
a motion sensor configured to sense motion of the subject and generate a motion signal according to the sensed motion,
wherein the motion signal serves as one of the at least one sensing signal,
wherein the vital signs data comprises information related to a state of motion of the subject.

5. The physiological monitoring apparatus as claimed in claim 1, further comprising:
an oxygen-level measurement circuit configured to receive the first PPG signal and measure the blood oxygen level of the subject according to the first PPG signal to generate a blood saturation percentage.

6. The physiological monitoring apparatus as claimed in claim 1, wherein the specific event indicates an apnea event happening to the subject.

7. The physiological monitoring apparatus as claimed in claim 1,
wherein the first PPG sensor comprises an infrared light source and a red light source, and
wherein in response to the PPG controller activating the first PPG sensor, the infrared light source and the red light source emit light beams.

8. The physiological monitoring apparatus as claimed in claim 1, further comprising:
a pre-processor configured to receive the at least one sensing signal and process the at least one sensing signal by filtering a direct-current component and high-frequency noise from the at least one sensing signal and removing at least one signal section, which corresponds to motion artifact of the subject, from the at least one sensing signal,
wherein the pre-processor outputs the at least one sensing signal which has been processed to the vital signs detector, and the vital signs detector obtains the vital signs data according to the at least one sensing signal which has been processed.

9. The physiological monitoring apparatus as claimed in claim 1, wherein in response to activating the first PPG sensor for a predetermined period of time, the first PPG sensor is deactivated.

10. The physiological monitoring apparatus as claimed in claim 1, wherein in response to detecting the specific event, the first PPG sensor is deactivated at a time point near a minimum value of the blood oxygen level.

11. A physiological monitoring method comprising:
sensing at least one physiological feature of a subject to generate at least one sensing signal;
obtaining vital signs data according to the at least one sensing signal;
detecting whether a specific event is happening to the subject according to the vital signs data;
in response to detecting that the specific event is happening to the subject, activating a PPG sensor to sense pulses of a blood vessel of the subject and to generate a first PPG signal according to the sensed pulses; and
obtaining a blood oxygen level of the subject according to the first PPG signal.

12. The physiological monitoring method as claimed in claim 11, wherein sensing the at least one physiological feature of the subject further comprises:
sensing pulses of a blood vessel of the subject to generate a second PPG signal,
wherein the second PPG signal serves as one of the at least one sensing signal, and
wherein the vital signs data comprises information related to at least one of a heart rate of the user, a respiration rate, and breathing activity of the subject.

13. The physiological monitoring method as claimed in claim 11, wherein sensing the at least one physiological feature of the subject further comprises:
sensing electrical activity of the heart of the subject to generate an ECG signal,
wherein the ECG signal serves as one of the at least one sensing signal, and
wherein the vital signs data comprises information related to a heart rate of the subject.

14. The physiological monitoring method as claimed in claim 12, wherein sensing the at least one physiological feature of the subject further comprises:
sensing motion of the subject to generate a motion signal according to the sensed motion,
wherein the motion signal serves as one of the at least one sensing signal,
wherein the vital signs data comprises information related to a state of motion of the subject.

15. The physiological monitoring method as claimed in claim 11, further comprising:
generating a blood saturation percentage according to the obtained blood oxygen level.

16. The physiological monitoring method as claimed in claim 11, wherein the specific event indicates an apnea event happening to the subject.

17. The physiological monitoring method as claimed in claim 11,
wherein the PPG sensor comprises an infrared light source and a red light source, and
wherein in response to activating the first PPG sensor, the infrared light source and the red light source emit light beams.

18. The physiological monitoring method as claimed in claim 11, further comprising:
processing the at least one sensing signal by performing a filter operation and a motion-artifact removal operation on the at least one sensing signal,
wherein the vital signs data is obtained according to the at least one sensing signal which has been processed.

19. The physiological monitoring method as claimed in claim 11, further comprising:
in response to activating the first PPG sensor for a predetermined period of time, deactivating the PPG sensor.

20. The physiological monitoring method as claimed in claim 11, further comprising:
in response to detecting the specific event, deactivating the first PPG sensor at a time point near a minimum value of the blood oxygen level.
